(19) United States
(12) Patent Application Publication
(10) Pub. No.: US 2005/0015263 A1
(43) Pub. Date: Jan. 20, 2005

(54) Use of gingko biloba extracts to promote neuroprotection and reduce weight loss

(76) Inventors: M Flint Beal, New York, NY (US); Robert J. Ferrante, Canton, MA (US)

Correspondence Address: Dinesh Agarwal;Law Office, 5350 Shawnee Road, Suite 330, Alexandria, VA, 22312, US

(21) Appl. No.: 10/495,335

(22) PCT Filed: Nov. 27, 2002
(86) PCT No.: PCT/US02/37988

Related U.S. Application Data
(60) Provisional application No. 60/334,442, filed on Nov. 29, 2001.

(51) Int. Cl. A61K 47/00; G06F 17/60

(52) U.S. Cl. 705/1; 424/439

(57) ABSTRACT

The invention is directed to methods of preventing, delaying, or reducing motor neuron damage in an individual by administering to the individual a composition containing an extract of gingko biloba. The methods can be used to treat an individual having or at risk of having a condition characterized by motor neuron damage, e.g., amyotrophic lateral sclerosis. The invention also includes methods of preventing or reducing weight loss in an individual by administering to the individual a composition containing an extract of gingko biloba.

### FIELD OF THE INVENTION

The invention relates to extracts of gingko biloba and their use in promoting neuroprotection and in preventing or reducing weight loss.

### BACKGROUND

Amyotrophic lateral sclerosis (ALS) is a progressive neurodegenerative disorder characterized by a loss of both upper and lower motor neurons, resulting in progressive paralysis and premature death. Missense mutations in the enzyme copper/zinc superoxide dismutase (SOD1) are associated with 15-20% of autosomal dominant familial ALS cases (Rosen et al. (1993) Nature 362:59-62). The superoxide dismutases are a family of enzymes that play a crucial role in the protection of oxygen radical-induced cellular damage. More than 60 mutations in SOD1 have been found to be associated with familial ALS (FALS).

Two hypotheses have been made concerning a potential aberrant gain of function of the mutant SOD1 enzyme. The first is that the mutant enzyme has an altered substrate affinity, leading to the generation of toxic reaction products (Beckman et al. (1993) Nature 364:584). In particular, it has been proposed that the mutant enzyme may more readily react with hydrogen peroxide or produce peroxynitrite (Estevez et al. (1999) Science 286:2498-2500). The mutant enzyme has a lowered affinity for zinc and can be more readily reduced by intracellular antioxidants such as ascorbate (Crow et al. (1997) J. Neurochem. 69:1945-1953); Lyons et al. (1996) Proc. Natl. Acad. Sci. USA 93:12240-12244). It can subsequently react to generate superoxide, as well as peroxynitrite, within the active site of the enzyme (Estevez et al., supra). Peroxynitrite can mediate nitration of tyrosines and cause oxidative damage to proteins, lipids, and DNA (Beckman et al. (1990) Proc. Natl. Acad. Sci. USA 87:1620-1624).

A second hypothesis as to the toxic mechanism of the mutant enzyme proposes that SOD1 forms intracellular aggregates. Transgenic mouse models of ALS have demonstrated cytoplasmic aggregates that stain with SOD1 and ubiquitin antibodies (Bruijn et al. (1998) Science 281:1851-1854). The formation of cytoplasmic inclusions and astrocytes in G85R transgenic ALS mice is a prominent pathologic feature (Bruijn et al., supra). In cultures of spinal neurons, the expression of a mutated SOD1 cDNA results in the formation of cytoplasmic aggregates (Durham et al. (1997) J. Neuropath. Exp. Neurol. 56:523-530), which leads to apoptotic cell death. These observations raise the possibility that the protein aggregates may be exerting a toxic effect. There is also evidence that mitochondrial dysfunction plays a prominent role in the pathogenesis of neuronal degeneration in the transgenic mouse models of ALS (kong and Xu (1998) J. Neurosci. 18:3241-3250).

### SUMMARY OF THE INVENTION

The invention is based, at least in part, on the discovery that an extract of gingko biloba exerts a neuroprotective effect, increases lifespan, and delays or decreases the development of clinical and neuropathologic symptoms in an animal model of ALS. The invention is also based on the discovery that an extract of gingko biloba decreases the extent of weight loss associated with the animal model of ALS. The present invention includes methods of preventing, delaying, or reducing motor neuron damage in an individual by administering to the individual a composition containing an extract of gingko biloba. Also included in the invention are methods of preventing or reducing weight loss in an individual by administering to the individual a composition containing an extract of gingko biloba.

In one aspect, the invention features a method of preventing or reducing weight loss in an individual. The method includes the steps of: selecting an individual having or at risk of having a condition characterized by weight loss; and administering to the individual a composition containing an extract of gingko biloba, wherein the administration prevents or reduces weight loss in the individual.

In one embodiment, the individual has a neurodegenerative disease. For example the individual can have a neurodegenerative disease such as amyotrophic lateral sclerosis that is characterized by damage to motor neurons.

In another embodiment, the individual has a cancer.

In another embodiment, the individual has a viral disease, e.g., the individual is infected with the human immunodeficiency virus.

In another embodiment, the individual has an eating disorder, e.g., anorexia nervosa.

In one embodiment, the extract of gingko biloba contains bilobalide. For example, the extract of gingko biloba can contain Egb761. Egb761 can be administered to the individual in an amount between about 120 to 240 mg. For example, Egb761 can be administered to the individual orally in an amount between about 120 to 240 mg per day for at least one week.

In another aspect, the invention features a method of preventing, delaying, or reducing motor neuron damage in an individual. The method includes the steps of: selecting an individual diagnosed as having or as being at risk for having a condition characterized by motor neuron damage; and administering to the individual a composition containing an extract of gingko biloba, wherein the administration prevents, delays, or reduces motor neuron damage in the individual. In one example, the condition is characterized by upper and lower motor neuron damage.

In one embodiment, the individual is diagnosed as having or as being at risk for having amyotrophic lateral sclerosis. For example, the individual can have a mutation in the superoxide dismutase 1 (SOD1) gene and the method can include identifying this mutation in the individual.

In one embodiment, the administration of the composition delays the onset of symptoms of amyotrophic lateral sclerosis.

In one embodiment, the administration of the composition increases the expected lifespan of the individual.

In one embodiment, the administration of the composition prevents, reduces, or delays weight loss in the individual.

In one embodiment, the administration of the composition results in improved motor function in the individual.

In one embodiment, the administration of the composition decreases the rate or extent of neuronal loss in the individual.

In one embodiment, the extract of gingko biloba contains bilobalide. For example, the extract of gingko biloba can contain Egb761. Egb761 can be administered to the individual in an amount between about 120 to 240 mg. For example, Egb761 can be administered to the individual orally in an amount between about 120 to 240 mg per day for at least one week.

In another aspect, the invention features a kit containing an extract of gingko biloba and instructions for use to reduce or prevent weight loss.

In another aspect, the invention features a kit containing an extract of gingko biloba and instructions for use to reduce or prevent motor neuron damage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A-1B depict the effects of 0.022% and 0.045% EGb761 on cumulative survival in male (A) and female (B) G93A transgenic ALS mice.

FIGS. 2A-2B depict the effects of 0.022% EGb761 on weight loss in male (A) and female (B) G93A transgenic ALS mice. Unsupplemented mice are depicted in dark circles.

FIGS. 3A-3D depict the effects of 0.022% and 0.045% EGb761 on rotarod performance in male (A and B, 0.022% and 0.045% EGb761 respectively) and female (C and D, 0.022% and 0.045% EGb761 respectively) G93A transgenic ALS mice. Unsupplemented mice are depicted in dark circles.

### DETAILED DESCRIPTION

The present invention provides methods of administering to an individual a composition containing an extract of gingko biloba to prevent, delay, or reduce motor neuron damage in the individual. These methods can be used to treat conditions characterized by damage to motor neurons, such as ALS. The invention also includes methods of preventing or reducing weight loss in an individual by administering to the individual a composition containing an extract of gingko biloba.

As described in the accompanying Examples, an extract of gingko biloba has been found to exert a neuroprotective effect, increase lifespan, decrease weight loss, and delay or decrease the development of clinical and neuropathologic symptoms in an animal model of ALS. Accordingly, pharmaceutical compositions containing an extract of gingko biloba can be used to treat conditions characterized by such features.

Gingko Biloba Containing Compounds

The invention comprises methods of administering to an individual a composition comprising an extract of gingko biloba.

The term “extract of ginkgo biloba” as used herein includes a collection of natural molecules (or pharmaceutically active derivatives thereof), including ginkgo terpenoids, derived from the Ginkgo biloba tree.

The term “ginkgo terpenoid” as used herein includes the naturally occurring terpenes that are derived from the gymnosperms tree Ginkgo biloba, as well as synthetically produced ginkgo terpenoids and pharmaceutically active derivatives and salts thereof and mixtures thereof. Examples of ginkgo terpenoids include ginkgolides and bilobalide. Examples of ginkgo terpenoids are disclosed in Ginkgolides, Chemistry, Biology, Pharmacology, and Clinical Perspectives, J. R. Provs. Science Publishers, Edited by P. Braguet (1988); F V. DeFeudis, Ginkgo Biloba Extract (Egb761); Pharmacological Activities and Clinical Applications, Elsevier, Chapter 11 (1991).

The terms “ginkgolide” and “bilobalide” as used herein include the various ginkgolides and bilobalide disclosed in the references cited above as well as non-toxic pharmaceutically active derivatives thereof. Examples of ginkgolide and bilobalide derivatives include tetrahydro derivatives, acetyl derivatives, and alkyl esters such as the monoacetate derivatives and triacetate derivatives disclosed in Okabe, et al., J. Chem. Soc. (c), pp. 2201-2206 (1967) and WO 99/64028.

Preferably, the extract is the ginkgo biloba extract EGb761. EGb761 is a standardized extract of green ginkgo biloba leaves and is a complex chemical mixture. It contains 24% flavonol glycosides, 6% terpene trilactones substances (ginkgolides and bilobalide), proanthocyanidins, and organic acids. EGb761 is described in detail in Ginkgo biloba Extract (EGb 761) Pharmacological Activities and Clinical Applications, DeFeudis, F. V., Eds, Elsevier, 1991; and Ullstein Medical 1998, Gingko biloba extract (EGb 761) Eds. Wiesbaden, DeFeudis, F. V.

The methods of the invention also include administering to an individual a composition containing a synthetically produced component (or a pharmaceutically active derivative of a component) of an extract of gingko biloba. For example, a composition can contain ginkgolide, bilobalide, or a derivative of ginkgolide or bilobalide.

Pharmaceutical Compositions and Methods of Administration

Pharmaceutical compositions for use in accordance with the present invention can be formulated in a conventional manner using one or more physiologically acceptable carriers or excipients. Thus, the compounds and their physiologically acceptable salts and solvates may be formulated for oral, buccal, parenteral, or rectal administration, or administration by inhalation, insufflation (either through the mouth or the nose).

For oral administration, the pharmaceutical compositions may take the form of, for example, capsules, tablets, pills, powders or granules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). Tablets and pills can additionally be prepared with enteric coatings.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. Alternatively, the active ingredient can be in powder form for constitution with a suitable vehicle, for example, sterile pyrogen-free water, before use.

The compounds can also be formulated in rectal compositions such as suppositories or retention enemas, for example, containing conventional suppository bases such as cocoa butter or other glycerides.

In addition to the formulations described previously, the compounds may also be formulated as a depot preparation. Such long acting formulations may be administered by implantation (e.g., subcutaneously or intramuscularly) or by intramuscular injection. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The therapeutic compositions of the invention can also contain a carrier or excipient, many of which are known to persons of ordinary skill in the art. Excipients that can be used include buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, sorbitol, and glycerol.

The dosage of gingko biloba extract contained in the composition can vary depending upon the desired therapeutic effect, the route of administration, and the duration of the treatment. However, it is necessary that the amount of the active ingredient be such that a suitable dosage form is obtained, e.g., a dose that causes the neuroprotective and/or weight related effects described herein. The dose can be administered as a single dose or divided into multiple doses.

In one example, Egb761 is administered to an individual in an amount between about 50 to 1,000 mg, 100 to 500 mg, or 120 to 240 mg. In a preferred embodiment, Egb761 is administered to the individual orally in an amount between about 120 to 240 mg per day for at least one week

In another example, a composition containing bilobalide is administered to an individual. Bilobalide may be administered in an amount of 0.05 to 2 mg/kg body weight of the individual or, preferably, administered in an amount of 0.1 to 1 mg/kg body weight of the individual.

Reducing or Preventing Damage to Motor Neurons

As described herein, a composition containing an extract of gingko biloba can be used to prevent, delay, or reduce motor neuron damage in an individual. In general, the methods include steps of selecting an individual diagnosed as having or as being at risk for having a condition characterized by motor neuron damage, and administering to the individual a composition containing an extract of gingko biloba.

A variety of motor neuron diseases can be treated using the methods described herein. For example, the methods of the invention can be used to treat diseases characterized by upper and/or lower motor neuron damage. Examples of such diseases include ALS, primary muscular atrophy, spinal muscular atrophy, progressive muscular atrophy, progressive bulbar atrophy, and hereditary spastic paraplegias.

With respect to ALS, the methods of the invention can be used to treat familial and/or non-familial forms of the disease. For familial ALS, it may be particularly advantageous to administer a composition described herein before the onset of symptoms. For example, an individual can be diagnosed as having a mutation in the superoxide dismutase 1 (SOD1) gene that is associated with the development of ALS. By administering a composition containing an extract of gingko biloba to such an individual before the onset of symptoms of ALS, the treatment can delay the onset of symptoms and/or reduce the severity of symptoms when they do occur. In addition, such treatment can be used to extend the expected lifespan of an individual diagnosed as having ALS or as being susceptible to developing familial ALS. Beneficial effects of such a treatment can be detected by any of the methods described herein, e.g., by detecting improved motor function in the individual or by detecting the rate or extent of neuronal loss in the individual. Neuronal loss can be evaluated, for example, by using imaging techniques such as magnetic resonance imaging. In addition, administering a composition containing an extract of gingko biloba can have beneficial effects with respect to reducing the weight loss associated with the development of ALS. The use of such compositions to reduce or prevent weight loss is described in detail in the following section.

Reducing or Preventing Weight Loss

As described herein, a composition containing an extract of ginkgo biloba can be used to prevent or reduce weight loss in an individual. In general, the methods include steps of selecting an individual having or at risk of having a condition characterized by weight loss, and administering to the individual a composition comprising an extract of gingko biloba The methods of the invention can also include steps of weighing the individual before and/or after the treatment. The weighing after the commencement of the treatment can be at regular intervals, e.g., daily, weeldy, or monthly. In addition, the dosage of the composition administered to the individual can be adjusted based upon the results of weight measurements taken before and/or after the commencement of the treatment. For example, the dosage can be increased if excessive weight loss occurs following an initial administration of a composition described herein.

Any condition associated with excessive or undesirable weight loss or an undesirably low weight can be treated using the methods described herein. Such disorders include, but are not limited to, cancers, autoimmune disorders, viral diseases, neurodegenerative disorders, and eating disorders.

The term cancer includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Cancers of a variety of organ systems are associated with excessive or undesirable weight loss and can thus be treated with compositions described herein.

The methods described herein can also be used to treat a wide variety of eating disorders characterized by excessive or unwanted weight loss. One example of such a disorder is anorexia nervosa.

Viral diseases that can be treated according to the methods described herein include viral diseases associated with cachexia or a wasting syndrome, such as HIV infection. Neurodegenerative disorders associated with weight loss or wasting include Alzheimer's disease, Huntington's disease, Parkinson's disease, and ALS.

The invention will be further described in the following examples, which do not limit the scope of the invention described in the claims.

### EXAMPLES

### Example 1

Effect of Egb761 Administration on the Survival of G93A Mutant Transgenic Mice

Transgenic mice with the G93A human SOD1 mutation (G1H/+) were obtained from Jackson Laboratories (Bar Harbor, ME). Male G1H/+ mice were bred with female mice on the B6SJL background strain and the offspring were genotyped by PCR of DNA obtained from tail tissue. Twenty male and female mice from each feeding paradigm were fed with either an unsupplemented diet or a diet supplemented with 0.022% or 0.045% EGb761 (Beaufour Ipsen Pharma, Paris, France) started at 21 days of age. This corresponds to 200 mg/kg/d and 400 mg/kg/d, respectively. Mice were weighed weekly starting at 23 days of age and twice weekly starting at 90 days of age.

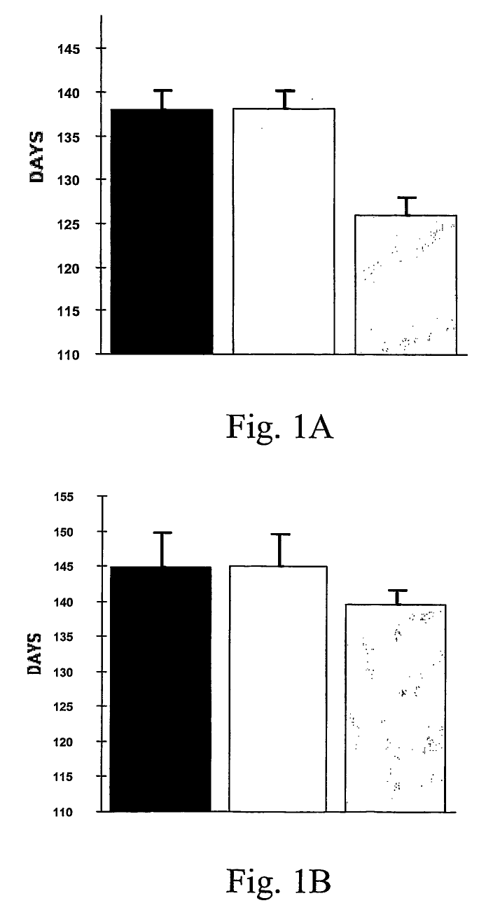

The oral administration of EGb761 resulted in a significant increase in survival in male transgenic G93A mice supplemented with either 0.022% (137.9±2.3 d) or 0.045% (138.2±1.9 d) Egb761 as compared to unsupplemented littermate G93A male mice (126.0±2.0 d) (p<0.001) (FIG. 1A). The increase in survival was less pronounced in female transgenic G93A mice supplemented with either 0.022% (144.9±4.8) or 0.045% (145±4.6) Egb761 as compared to unsupplemented littermate G93A female mice (139.6±2.2) (FIG. 1B).

Statistical comparisons for survival were made using the Mantel-Cox log-rank test. Statistical comparisons of other parameters were made by analysis of variance (ANOVA) or repeated measures ANOVA of other parameters followed by the Fisher Least Significant Difference test.

### Example 2

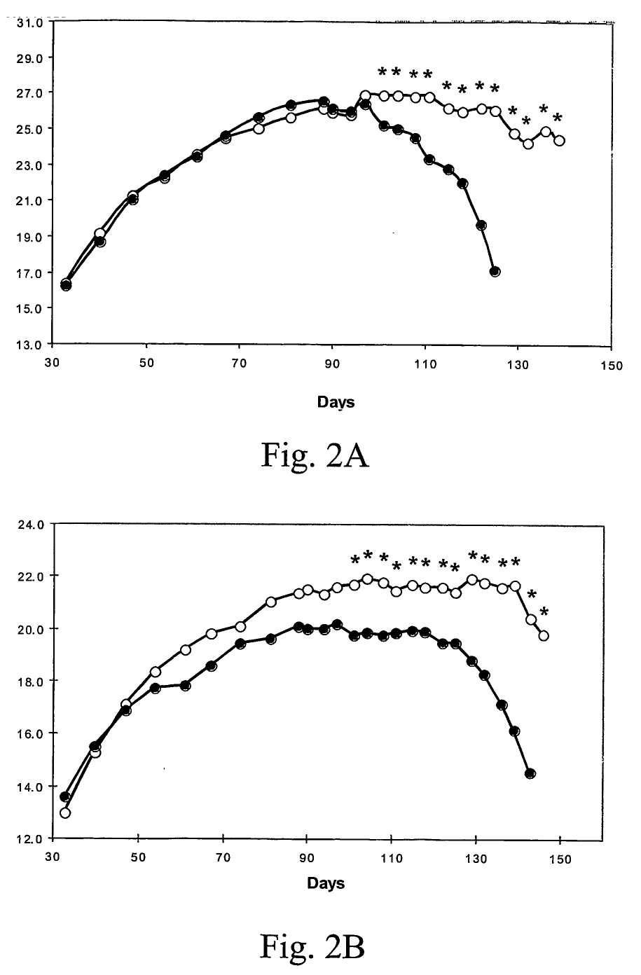

Effect of Egb761 Administration on the Age-Dependent Loss of Bode Weight in G93A Mutant Transgenic Mice In both male and female transgenic G93A mice, oral administration of EGb761 significantly delayed an age-dependent loss of body weight. The effects of oral administration of EGb761 on body weight in G93A transgenic mice are shown in FIGS. 2A-2B. Both EGb761 regimens (0.022% and 0.045%) resulted significant improvements of body weight as compared to unsupplemented G93A mice. While body weight measurements were recorded throughout the temporal sequence of the experiment in the 0.022% EGb761 treated G93A mice, significance was only found from 101 days in both male (FIG. 2A) and female (FIG. 2B) mice, as compared to unsupplemented G93A mice. Unsupplemented mice are depicted in dark circles in FIGS. 2A-2B (*p<0.05).

### Example 3

Effect of Egb761 Administration on Muscle Strength in G93A Mutant Transgenic Mice

Performance on rotarod as an index of muscle strength was assessed weekly starting at 23 days of age and twice weekly starting at 90 days of age. Mice were given two days to become acquainted with the rotarod apparatus (Columbus Instruments, Columbus, Ohio). The rotarod was maintained at 10 rpm. Each mouse was given three trials at 60 seconds each for a maximum of 180 seconds at each time point. The length of time at which the mouse fell off the rotating rod was used as the measure of competency on this task. Mice were tested until they were unable to perform the task (120 days and 130 days for male and female mice, respectively). Mice were euthanized when they were no longer able to right themselves within 30 seconds of being placed on their sides. This time point was used as the time of death.

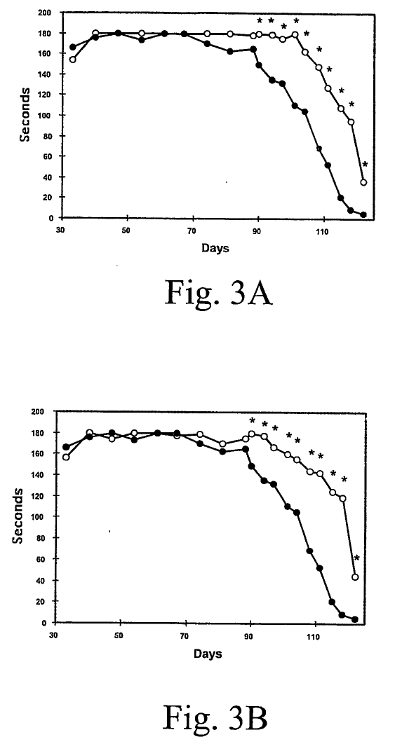

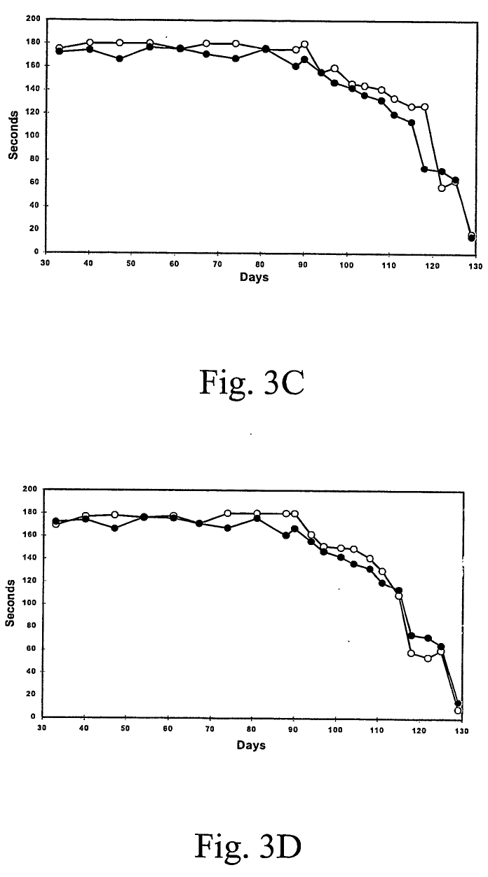

The effects of oral administration of 0.022% and 0.045% EGb761 on rotarod performance between 30 and 130 days are shown in FIGS. 3A-3D. Administration of both 0.022% and 0.045% EGb761 significantly improved rotarod performance in male G93A mice after 90 days of age, as compared to unsupplemented male G93A mice (FIGS. 3A and 3B, respectively). A similar effect was not observed in female mice supplemented with 0.022% and 0.045% EGb761 (FIGS. 3C and 3D, respectively). Unsupplemented mice are depicted in dark circles (*p<0.05).

### Example 4

Effect of Egb761 Administration on Neuronal Loss in G93A Mutant Transgenic Mice

Transgenic G93A male and female mice administered 0.022% and 0.045% EGb761 and wild type littermate mice were analyzed for histopathologic changes. Groups of 10 animals were deeply anesthetized and then transcardially perfused with 4% buffered paraformaldehyde at 120 and 134 days, for male and female mice respectively. There is a sexual dymorphism in familial ALS mice such that mortality is earlier in males than in females (Trieu and Uckun (1999) Biochem. Biophys. Res. Comm. 258:685-688). The spinal cords were removed from the mice, post-fixed with the perfusant for 2 hours, cryoprotected in a graded series of 10% and 20% glycerol/2% DMSO solution, and subsequently serially frozen sectioned at 50 um, stored in 6 well tissue collection clusters, and stained for Nissl substance (cresyl violet).

Serial-cut coronal tissue-sections from the cervical 4-5 segments of the spinal cord were used for neuronal analysis. Unbiased stereologic counts of Nissl stained neurons were obtained from the anterior horn of the cervical spinal cord from unsupplemented and 0.022% and 0.045% EGb761 supplemented G93A mice, as well as littermate transgene negative mice, using Neurolucida Stereo Investigator software (Microbrightfield, Colchester, Vt.). The ventral horn was delineated by a line from the central canal laterally and circumscribing the belly of gray matter to include spinal cord layers 7-9. The total area of the ventral horn was defined in 20 serial sections from each spinal cord specimen in which counting frames were randomly sampled. The dissector counting method was employed in which Nissl stained neurons were counted in an unbiased selection of serial sections within the cervical spinal cord. All computer identified cell profiles were manually verified as neurons. Data is represented as percent change.

Nissl stained sections of the cervical spinal cord in 0.022% EGb761 supplemented and unsupplemented male and female G93A mice showed marked neuronal loss of ventral horn neurons in comparison to wild type littermate control mice. Visual comparison suggested that the neuronal loss in the EGb761 supplemented G93A male mice was less prominent than in untreated male and treated female G93A mice.

These observations were confirmed using stereologic analysis of neuronal counts in the ventral horn. Both unsupplemented and 0.022% EGb761 supplemented male and female G93A transgenic mice showed significant neuronal loss, as compared to wild type littermate control mice (Table 1). The data presented in Table 1 are mean±SEM per thousand neurons.

TABLE 1
Neuronal Counts in ALS Transgenic Mice

| | Male | Female |
|---|---|---|
| Wild Type | 6.495 ± 0.11 | 6.432 ± 0.143 |
| G93A | 1.688 ± 0.219 | 1.865 ± 0.244 |
| G93A − EGb761 | 2.727 ± 0.207 | 2.058 ± 0.239 |

The percent differences in ventral horn neuron-loss in the EGb761 supplemented and unsupplemented female G93A mice were 68±5.3% and 71±7.2%, respectively, as compared to wild type littermate control mice (Table 1). The percent differences in ventral horn neuron-loss in the EGb761 supplemented and unsupplemented male G93A mice were 56±5.3% and 74±6.9%, respectively, as compared to wild type littermate control mice (Table 1). Thus, significantly less neuronal loss was found in the 0.022% EGb761 supplemented male G93A mice, as compared to unsupplemented G93A male mice (p<0.001), as well as to 0.022% supplemented female G93A mice (p<0.01). No significant differences in cervical, ventral-horn neuronal number were present between male and female wild type littermate control mice.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preventing or reducing weight loss in an individual, the method comprising:
selecting an individual having or at risk of having a condition characterized by weight loss; and
administering to the individual a composition comprising an extract of ginkgo biloba,
wherein the administration prevents or reduces weight loss in the individual.

2. The method of claim 1, wherein the individual has a neurodegenerative disease.

3. The method of claim 2, wherein the neurodegenerative disease is characterized by damage to motor neurons.

4. The method of claim 3, wherein the neurodegenerative disease is amyotrophic lateral sclerosis.

5. The method of claim 1, wherein the individual has cancer.

6. The method of claim 1, wherein the individual has a viral disease.

7. The method of claim 6, wherein the individual is infected with the human immunodeficiency virus.

8. The method of claim 1, wherein the individual has an eating disorder.

9. The method of claim 8, wherein the individual has anorexia nervosa.

10. The method of claim 1, wherein the extract of gingko biloba comprises bilobalide.

11. The method of claim 1, wherein the extract of gingko biloba comprises Egb761.

12. The method of claim 11, wherein Egb761 is administered to the individual in an amount between about 120 to 240 mg.

13. The method of claim 12, wherein Egb761 is administered to the individual orally in an amount between about 120 to 240 mg per day for at least one week.

14. A method of preventing, delaying, or reducing motor neuron damage in an individual, the method comprising:
selecting an individual diagnosed as having or as being at risk for having a condition characterized by motor neuron damage; and
administering to the individual a composition comprising an extract of ginkgo biloba,
wherein the administration prevents, delays, or reduces motor neuron damage in the individual.

15. The method of claim 14, wherein the condition is characterized by upper and lower motor neuron damage.

16. The method of claim 14, wherein the individual is diagnosed as having or as being at risk for having amyotrophic lateral sclerosis.

17. The method of claim 16, wherein the individual has a mutation in the superoxide dismutase 1 (SOD1) gene.

18. The method of claim 17, wherein the administration delays the onset of symptoms of amyotrophic lateral sclerosis.

19. The method of claim 14, wherein the administration increases the expected lifespan of the individual.

20. The method of claim 14, wherein the treatment prevents, reduces, or delays weight loss in the individual.

21. The method of claim 14, wherein the administration results in improved motor function in the individual.

22. The method of claim 14, wherein the administration decreases the rate or extent of neuronal loss in the individual.

23. The method of claim 14, wherein the extract of gingko biloba comprises bilobalide.

24. The method of claim 14, wherein the extract of gingko biloba comprises Egb761.

25. The method of claim 24, wherein Egb761 is administered to the individual in an amount between about 120 to 240 mg.

26. The method of claim 25, wherein Egb761 is administered to the individual orally in an amount between about 120 to 240 mg per day for at least one week.

27. A kit comprising an extract of ginkgo biloba and instructions for use to reduce or prevent weight loss.

28. A kit comprising an extract of ginkgo biloba and instructions for use to reduce or prevent motor neuron damage.
